(19) United States
(12) Patent Application Publication
(10) Pub. No.: US 2002/0009506 A1
(43) Pub. Date: Jan. 24, 2002

(54) Compositions and methods for treating brain disorders and enhancing brain function

(76) Inventor: Yuanjin Tao, Fremont, CA (US)

Correspondence Address: Gladys H. Monroy, Morrison & Foerster LLP, 755 Page Mill Road, Palo Alto, CA, 94304-1018, US

(21) Appl. No.: 09/872,374

(22) Filed: Jun. 1, 2001

Related U.S. Application Data
(60) Provisional application No. 60/208,984, filed on Jun. 1, 2000.

(51) Int. Cl. A61K 35/78

(52) U.S. Cl. 424/728; 424/736; 424/756; 424/757; 424/752

(57) ABSTRACT

Compositions and methods for treating sub-optimal brain function or enhancing brain function are provided. The compositions comprise singly or in combination herbals, natural nutritional supplements, minerals and vitamins. Methods of making these compositions are also provided.

### CROSS-REFERENCE TO RELATED APPLICATIONS

This application claims the benefit of U.S. Provisional application Ser. No. 60/208,984, filed Jun. 1, 2000, hereby incorporated in its entirety by reference.

### TECHNICAL FIELD

This invention relates to compositions and methods from treating brain disorders and improving brain function. More specifically, it relates to nutraceutical compositions for treating sub-optimal brain function symptoms or enhancing brain function.

### BACKGROUND ART

Today's competitive work environments constantly challenge workers' intellect, and career advancements are increasingly dependent on one's productivity, which in turn relies in large part on a healthy and keen mind. Therefore, sub-optimal brain function symptoms, such as lack of mental alertness or concentration, absentmindedness and fatigue, can be problems with serious consequences.

A nutraceutical composition for invigorating and refreshing the intellect is known (Intellex Mind Sharpener, Amino Food Technologies, San Jose, Calif.). The ingredients in this formula include beta-carotene, natural vitamin E, ascorbic acid, thiamin, riboflavin, vitamin B-6, vitamin B-12, zinc, trunsing extract, siberian ginseng extract, chinese red ginseng extract, ginkgo biloba extract, ginger extract, quercetin, inositol and magnesium stearate.

A composition that improves mental well-being and mental acuity, and assists in relieving depression is known. U.S. Pat. No. 5,820,867. The ingredients include St. John's Wort extract, ginkgo biloba extract, vitamin B6, vitamin B12, folic acid and vitamin C.

A composition thought to increase blood flow to the brain to increase brain alpha rhythms, which are associated with mental alertness, is also known. U.S. Pat. No. 5,569,458. The composition consists of 70 different components (vitamins, minerals and herbs), one of which is ginkgo biloba.

A composition that improves mental functions, and treats memory impairment and disorientation is known. U.S. Pat. No. 4,892,883. This composition includes flavone glycosides and bilobalide.

In view of the importance of optimal functioning of the brain in today's society, and the corresponding advantage in alleviating conditions of sub-optimal brain function and enhancing brain function, it would be beneficial to have compositions and methods that are efficacious and safe to provide this advantage. The invention described and claimed in this specification presents such compositions and methods.

All references cited herein, including patent applications and publications, are incorporated by reference in their entirety.

### DISCLOSURE OF THE INVENTION

Compositions and methods for treating sub-optimal brain function or enhancing brain function are provided. The compositions comprise substances in quantities that are effective in treating sub-optimal brain function conditions or enhancing brain function. Methods of treating sub-optimal brain function or enhancing brain function comprising administration of the claimed compositions are also provided. Methods of making compositions of the invention are also provided.

Accordingly, in one aspect, the invention provides a composition for treating sub-optimal brain function or enhancing brain function comprising 1 substance selected from the group consisting of a member of the botanical group Angelica, a member of the botanical group Codonopsis, a member of the botanical group Jujuba and a member of the botanical group Schisandra.

In another aspect, the invention provides a composition for treating sub-optimal brain function or enhancing brain function comprising 2 substances selected from the group consisting of a member of the botanical group Angelica, a member of the botanical group Codonopsis, a member of the botanical group Jujuba and a member of the botanical group Schisandra.

In another aspect, the invention provides a composition for treating sub-optimal brain function or enhancing brain function comprising 3 substances selected from the group consisting of a member of the botanical group Angelica, a member of the botanical group Codonopsis, a member of the botanical group Jujuba and a member of the botanical group Schisandra.

In another aspect, the invention provides a composition for treating sub-optimal brain function or enhancing brain function comprising a member of the botanical group Angelica, a member of the botanical group Codonopsis, a member of the botanical group Jujuba and a member of the botanical group Schisandra.

In one embodiment, the invention provides a composition selected from the group consisting of the compositions of the aspects of the invention described in the preceding paragraphs, further comprising a substance selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF. Thus, for example, a composition may comprise 1, 2 or 3 substances selected from the group consisting of a member of the botanical group Codonopsis, a member of the botanical group Schisandra and a member of the botanical group Jujuba, further comprising a substance selected from the group consisting of a member of the botanical group Panax, a member of the botanical group senticosus and a member of the botanical group Ligusticum.

In one embodiment, the invention provides a composition selected from the group consisting of the compositions of the aspects of the invention described in the preceding paragraphs, further comprising 2 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF. Thus, for example, a composition may comprise 1, 2 or 3 substances selected from the group consisting of a member of the botanical group Codonopsis, a member of the botanical group Schisandra and a member of the botanical group Jujuba, further comprising 2 substances selected from the group consisting of a member of the botanical group Panax, a member of the botanical group senticosus and a member of the botanical group Ligusticum.

In one embodiment, the invention provides a composition selected from the group consisting of the compositions of the aspects of the invention described in the preceding paragraphs, further comprising 3 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF. Thus, for example, a composition may comprise 1, 2 or 3 substances selected from the group consisting of a member of the botanical group Codonopsis, a member of the botanical group Schisandra and a member of the botanical group Jujuba, further comprising 3 substances selected from the group consisting of a member of the botanical group Panax, a member of the botanical group senticosus and a member of the botanical group Ligusticum.

In one embodiment, the invention provides a composition selected from the group consisting of the compositions of the aspects of the invention described in the preceding paragraphs, further comprising 4 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 5 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 6 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 7 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 8 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 9 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 10 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 11 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 12 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 13 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 14 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF..

In one embodiment, the invention provides a composition selected from the group consisting of the compositions of the aspects of the invention described in the preceding paragraphs, further comprising 15 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyirhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 16 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 17 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 18 substances selected from the group consisting of a member of the botanical group Panax, a member of the botanical group Astragalus, a member of the botanical group Senticosus, a member of the, botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 19 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 20 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group. Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 21 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 22 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Lignsticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 23 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 24 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 25 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising 26 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In one embodiment, the invention provides a composition selected from the group consisting of the compositions of the aspects of the invention described in the preceding paragraphs, further comprising a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In various embodiments of the compositions of the invention described in the preceding and following paragraphs, the member of the botanical group Angelica may be dangqui, the member of the botanical group Codonopsis may be dangsen, the member of the botanical group Jujuba may be suanzaore and/or the member of the botanical group Schisandra may be wuweizi. In some embodiments, the member of the botanical group Angelica may be dangqui, the member of the botanical group Codonopsis may be dangsen, the member of the botanical group Jujuba may be suanzaore, the member of the botanical group Schisandra may be wuweizi, the member of the botanical group Angelica may be dangqui, the member of the botanical group Codonopsis may be dangsen, the member of the botanical group Jujuba may be suanzaore, the member of the botanical group Schisandra may be wuweizi, the member of the botanical group Panax may be renshen, the member of the botanical group Astragalus may be huangqi, the member of the botanical group Senticosus may be ciwujia, the member of the botanical group Gynostemma may be jiaogulan, the member of the botanical group Polygala may be yuanzhi, the member of the botanical group Citrus reticulata may be chenpi, the member of the botanical group Zingiberis may be ginger, the member of the botanical group Glycyrrhiza may be gancao, the member of the botanical group ginkgo may be ginkgo, the member of the botanical group Ligusticum may be chuanxiong, and/or the member of the botanical group Acorus gramineus may be shichangpu.

Accordingly, in one aspect, the invention provides a composition for treating sub-optimal brain function or enhancing brain function comprising a substance selected from the group consisting of dangqui, dangsen, suanzaore and wuweizi.

In another aspect, the invention provides a composition for treating sub-optimal brain function or enhancing brain function comprising 2 substances selected from the group consisting of dangqui, dangsen, suanzaore and wuweizi.

In another aspect, the invention provides a composition for treating sub-optimal brain function or enhancing brain function comprising 3 substances selected from the group consisting of dangqui, dangsen, suanzaore and wuweizi.

In yet another aspect, the invention provides a composition for treating sub-optimal brain function or enhancing brain function comprising dangqui, dangsen, suanzaore and wuweizi.

In one embodiment, the invention provides a composition selected from the group consisting of the compositions of the aspects of the invention described in the preceding paragraphs, further comprising a substance selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 2 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 3 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 4 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 5 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 6 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 7 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 8 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 9 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 10 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 11 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 12 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 13 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 14 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 15 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 16 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 17 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 18 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 19 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 20 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 21 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 22 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 23 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 24 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 25 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising 26 substances selected from the group consisting of renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another embodiment, the invention provides a composition selected from the group consisting of the compositions of the aspects of the invention described in the preceding paragraphs, further comprising renshen, huangqi, ciwujia, jiaogulan, yuanzhi, chenpi, ginger, gancao, ginkgo, chuanxiong, shichangpu, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

In another aspect, the invention provides a method for treating sub-optimal brain function or enhancing brain function comprising administering to the individual an effective amount of a composition selected from the group consisting of the compositions of the aspects and embodiments of the invention described in the preceding and following paragraphs, whereby sub-optimal brain function is treated or brain function is enhanced.

In another aspect, the invention provides a method of making a composition for treating sub-optimal brain function or enhancing brain function, said method comprising combining at least two (preferably 2, 3 or 4) substances (preferably in an effective amount) selected from the group consisting of a member of the botanical group Angelica, a member of the botanical group Codonopsis, a member of the botanical group Jujuba and a member of the botanical group Schisandra. In some embodiments of these methods, said at least two substances are combined with at least one (preferably 1, 2, 3, 4, 5, 6, 7, 8, 9, 10, 11, 12, 13, 14, 15, 16, 17, 18, 19, 20, 21, 22, 23, 24, 25, 26, or 27) substance (preferably in an effective amount) selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF. In some embodiments, said combining is by mixing (such as by stirring, agitation or vibration). In some embodiments, the substances are packaged in the form of capsules, preferably in size “0”, “00”, “000”, “1”, “2”, “3” or “4.” In yet other embodiments, the substances are combined in powder form, preferably to at least 30%, 60%, or 90% mixture consistency, or to homogeneity.

### MODES FOR CARRYING OUT THE INVENTION

The present invention discloses compositions comprising single or multiple substances that are effective and safe for treating conditions of sub-optimal brain function or enhancing brain function. The invention further provides methods of treating sub-optimal brain function or enhancing brain function using the disclosed compositions. Methods of making compositions of the invention are also provided.

Definitions

The term “treating,” “treatment,” and variations thereof, as used in this specification, refers to an approach for obtaining beneficial or desired physiological results, which may be established clinically. For purposes of this invention, beneficial or desired clinical results include, but are not limited to, alleviation of symptoms, diminishment of extent of disease, stabilized (i.e., not worsening) condition, delay or slowing of progression or worsening of condition/symptoms, amelioration or palliation of the condition or symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering compositions of the present invention.

A “treatment effect” or “therapeutic effect” is manifested if there is a change in the condition being treated, as measured by the criteria constituting the definition of the terms “treating” and “treatment.” There is a “change” in the condition being treated if there is at least 10% improvement, preferably at least 25%, more preferably at least 50%, even more preferably at least 75%, and most preferably at least 100%. The change can be based on improvements in the severity of the treated condition in an individual, or on a difference in the frequency of improved conditions in populations of individuals with and without treatment with the compositions of the present invention.

“Sub-optimal brain function,” as used herein, refers to symptoms or conditions involving the brain that prevent the optimal functioning or use of the brain. These symptoms or conditions can be those presently indicated as manifestations or any that an individual finds to be non-conducive to the optimal or normal functioning of the brain. Such symptoms or conditions include, but are not limited to, poor memory, lack of concentration, mental fatigue, irritability, ill-temper, insomnia, depression and/or absentmindedness.

“Enhance brain function,” “enhancing brain function,” “enhancement of brain function,” and variations thereof, as used herein, refer to an increase in the brain function following administration of the compositions of this invention compared to brain function without the administration of said compositions. “An increase in the brain function” is manifested when there is an increase or improvement in mental activities, condition, or performance, as indicated by criteria such as, but not limited to, memory level, ability to concentrate, mental sharpness or lack of fatigue, lack or level of irritability, lack or level of ill-temperedness, quality of sleep, lack or level of depression, and/or lack or level of absentmindedness. Enhancement is achieved when there is at least a 10% increase in brain function after a composition of the present invention is administered compared to before administration. Preferably the increase is at least 25%, more preferably at least 50%, even more preferably at least 75%, most preferably at least 100%. Such an increase can be measured by any method known to the person of skill in the art, including, but not limited to, self-assessment by the individual using a scale to indicate degree of the symptoms, conditions, or brain function, assessment by a health care professional using evaluation scales known in the art, and/or biochemical indicators known to correlate or be associated with manifestations of brain functions.

An “effective amount” is an amount of a composition or substance(s) sufficient to effect beneficial or desired results in the treatment of sub-optimal brain function or in the enhancement of brain function after one or more administrations of that amount. An effective amount can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

“Individual,” as used herein, refers to a vertebrate, preferably a mammal, more preferably a human.

A “botanical group,” as used herein, refers to a group of botanical entities that are capable of providing similar physiological effect(s) in the compositions of the invention. These botanical entities may or may not belong to the same botanical classification (such as genus, family).

“Dangquiwei,” or “Dangqui,” as used herein, refers to extracts of dangquiwei. It is also known as extremitas radix angelicae sinensis and angelica sinensis (Oliv) Diels. A member of the botanical group Angelica is a substance that is capable of providing a similar physiological effect(s) as that provided by Dangquiwei in the compositions of the invention, and is preferably selected from a group comprising Angelica sinensis (Oliv.) Diels.; Angelica acutiloba (s.et.Z.) Kitag; Angelica polymorpha Maxim. Var. sinensis Oliv.; Angelica polymorpha sinensis.; Angelica polymorpha sinensis; Angelica polymorpha sinensis (Oliv.); Angelica. polymorpha var. sinensis; Levisticum officinale Koch; Ligusticum acutilobum S.et Z.

“Dangsen,” as used herein, refers to extracts of dangsen. It is also known as radix codonopsitis pilosulae and codonopsis pilosula (Franch.) Nannf. A member of the botanical group Codonopsis is a substance that is capable of providing a similar physiological effect(s) as that provided by dangsen in the compositions of the invention, and is preferably selected from a group comprising Radix codonopsitis pilosulae; Codonopsis pilosula (Franch) Bge; Codonopsis pilosula (Franch) Nannf; Codonopsis tangshen Oliv.; Codonopsis pilosula tubulosa Komar.; Codonopsis viridiflora Maxim.; Codonopsis nervosa(Chiff.) Nannf.; Codonopsis tsinlingensis Pax.et.Hoffm; Codonopsis Clematidea Clarke.; Codonopsis lanceolata Benth. et Hook.; Codonopsis subglobosa W. W. Smith; Codonopsis canescens Nannf; Codonopsis cordiophy-Ha Diels; Codonopsis subscoposa Komar.; Codonopsis micrantha Chipp.; Codonopsis ovata Benth; Codonopsis macrocalyx Diels; Campanumoea pilosula. Franch.; Campanumoea pilosula.

“Suanzaore,” as used herein, refers to extracts of suanzaore. It is also known as semen zizphi spinosae and ziziphus spinosa hu. A member of the botanical group jujuba is a substance that is capable of providing a similar physiological effect(s) as that provided by suanzaore in the compositions of the invention, and is preferably selected from a group comprising Semen zizphi spinosulae; Zizphi spinosulae Hu; Zizyphus jujuba Mill var.spinosa Hu; Zizyphus jujuba Mill var.spinosa Bunge; Zizyphus jujuba spinosa (Bunge) Hu; Rhamnus jujuba; Rhamnus zizyphus ; Zizyphus mauritiana; Zizyphus spinosa; Zizyphus vulgaris var. spinosa; Zizyphus jujuba; Zizyphus lotos; Zizyphus sativa; Zizyphus vulgaris; Zizyphus zizyphus.

“Wuweizi,” as used herein, refers to extracts of wuweizi. It is also known as fructus schisandrae chinensis and (Turcz.) Baill. A member of the botanical group Schisandra is a substance that is capable of providing a similar physiological effect(s) as that provided by wuweizi in the compositions of the invention, and is preferably selected from a group comprising Fructus schisandrae chinensis; Schisandra chinensis (Turcz.)Baill; Schisandra sphenanthera Rehd et Wils; Schisandra henryi Clarks; Schisandra sphaerandra Stapf.; Schisandra japonica ((Siebold.&Zucc. ex A. Gray.)Hance.); Schisandra japonica; Maximowiczia chinensis ((Turcz.)Rupr.); Maximowicza chinensis; Sphaerostema j aponica; Sphaerostema japonicum; Kadsura chinensis; Kadsura chinensis (Turcz.); Renshen; Radix ginseng; Panax ginseng C. A. Mey; Panax schin-seng Nees; Panax schinseng.; Panax schinseng; Panax pseudoginseng; Panax pseudoginseng japonicus (C. A. Mey.)Hoo.&Tseng.; Panax repens. Max.; Panax pseudoginseng japonicus; Panax pseudoginseng ssp. Japonicus; Panax pseudoginseng subsp.; Panax japonicus; Panax pseudoginseng var. japonicus; Panax pseudo-ginseng; Panax ginseng; Panax schinseng.

“Renshen,” as used herein, refers to extracts of renshen. It is also known as radix ginseng and panax ginseng bC. A. Mey. A member of the botanical group Panax is a substance that is capable of providing a similar physiological effect(s) as that provided by renshen in the compositions of the invention, and is preferably selected from a group comprising Radix notoginsheng; Panax notoginsheng (Burk.) F. H. Chen.; Panax notoginseng (Burk.) F. H. Chen (Shanqi); Panax pseudo-ginseng Wall.var.notoginseng (Burk) Hoo et Tseng; Panax ginseng C. A. Mey (P.schinseng Nees); Panax guinquefolium L.; Radix ginseng; Panax ginseng C. A. Mey; Panax schin-seng Nees; Panax schinseng.; Panax schinseng; Panax pseudoginseng; Panax pseudoginsengjaponicus (C. A. Mey.)Hoo.&Tseng.; Panax repens. Max.; Panax pseudoginseng japonicus; Panax pseudoginseng ssp. Japonicus; Panax pseudoginseng subsp.; Panax japonicus; Panax pseudoginseng var. japonicus; Panax pseudo-ginseng; Panax ginseng; Panax schinseng Acanthopanax sentocosus (Rupr. et Maxim.); Eleutherococus senticosus (Rupr. et Maxim.).; Eleutherococus senticosus; Acanthopanax senticosus; Acanthopanax seticosus Harms; Acantherococus senticocus (Rupr.et Maxim) Harms.; Eleutherococus senticocus(Rupr. Et Maxim) Maxim.; Echinopanax elatus Nakai; Hedera senticosa.

“Huangqi,” as used herein, refers to extracts of hungqi. It is also known as radix astragali membranaceus and astragalus membranaceus (Fisch) Bge. A member of the botanical group Astragalus is a substance that is capable of providing a similar physiological effect(s) as that provided by huangqi in the compositions of the invention, and is preferably selected from a group comprising Radix astragali membranaceus; Astragalus membranaceus (Fisch)Bge; Astragalus membranaceus; Astragalus mongholicus Bunge; Astragalus tongolensis Ulbr.; Astragalus chrysopterus Bunge; Astragalus floridus Benth; Astragalus maonwenensis Hsiao; Astragalus yunnanensis Franch; Astragalus ernestii Comb.; Hedysarum polybotrys Hand-Mazz; Hedysarum austrosibiricum B.; Hedysarun limprichtii Ulbr.; Hedysarum viciodes Turez var taipeicum (Hand-Mazz) Lin.; Hedysarum sikkimense Benth.

“Ciwujia,” as used herein, refers to extracts of ciwujia. It is also known as siberian ginseng and eleutherococcus senticosus. A member of the botanical group Senticosus is a substance that is capable of providing a similar physiological effect(s) as that provided by ciwujia in the compositions of the invention, and is preferably selected from a group comprising Eleutherococus senticosus; Acanthopanax senticosus; Acanthopanax seticosus Harms; Acantherococus senticocus (Rupr.et Maxim) Harms.; Eleutherococus senticocus(Rupr. Et Maxim) Maxim.; Echinopanax elatus Nakai; Hedera senticosa.

“Jiaogulan,” as used herein, refers to extracts of jiaogulan. It is also known as gynostemma pentaphyllum (Thunb.) Mak. A member of the botanical group Gynostemma is a substance that is capable of providing a similar physiological effect(s) as that provided by jiaogulan in the compositions of the invention, and is preferably selected from a group comprising Gynostemma pentaphyllum; Gynostemma pentaphyllum (thunb.)Mak.; Gynostemma pedatum; Vitis pentaphylla.

“Yuanzhi,” as used herein, refers to extracts of yuanzhi. It is also known as radix polugalae tenuifoliae and polygala tenuifolia willd. A member of the botanical group Polygala is a substance that is capable of providing a similar physiological effect(s) as that provided by yuanzhi in the compositions of the invention, and is preferably selected from a group comprising Radix polugalae tenuifoliae; Polygala tenuifoliae; Polygala japonica Houtt; Polygala sibirica L.

“Chenpi,” as used herein, refers to extracts of chenpi. It is also known as pericarpium citri reticulatae and citrus reticulata blanco. A member of the botanical group Citrus reticulata is a substance that is capable of providing a similar physiological effect(s) as that provided by chenpi in the compositions of the invention, and is preferably selected from a group comprising Pericurpium citri reticulatae; Citrus reticulata Blanco; Citrus reticulata Blanco var chachiensis. H. H. Hu.; Citrus reticulata Blanco var deliciosa. H. H. Hu.; Citrus reticulata Blanco var erythorosa. H. H. Hu.; Citrus reticulata Blanco var kinokuni.(Tanaka) H. H. Hu.; Citrus reticulata Blanco var Suavissima(Tanaka) H. H. Hu.; Citrus reticulata Blanco var Succosa(Tanaka) H. H. Hu; Citrus reticulata Blanco var tardiferax(Tanaka) H. H. Hu; Citrus reticulata Blanco var Unshin(Marcor) Suavissima(Tanaka) H. H. Hu; Citrus chachiensis Hort Citrus tangerina Hort et Tanaka Citrus nobilis.

“Ginger,” as used herein, refers to extracts of ginger. It is also known as rhizoma zingiberis officinalis recens and zingiber officinale Rosc. A member of the botanical group Zingiberis is a substance that is capable of providing a similar physiological effect(s) as that provided by ginger in the compositions of the invention, and is preferably selected from a group comprising Rhizoma zingiberis officinalis recens.; Zingiber officinale Rosc.

“Gancao,” as used herein, refers to extracts of gancao. It is also known as radix glycyrrhizae uralensis and glycyrrhiza uralensis Fischer. A member of the botanical group Glycyrrhiza is a substance that is capable of providing a similar physiological effect(s) as that provided by gancao in the compositions of the invention, and is preferably selected from a group comprising Radix glycyrrhizae uralensis; Glycyrrhiza uralensis Fischer; Glycyrrhiza uralensis Fisch (Gan cao); Glycyrrhiza glabra L.; Glycyrrhizainflata Batal.; Glycyrrihiza korshiskyi G. Hrig.; Glycyrrhiza aspera Pall; Glycyrrhiza yunnanensis Cheng f. et L. K. Tai.; Glycyrrhiza malensis.

“Ginkgo,” as used herein, refers to extracts of ginkgo. It is also known as ginkgo bilobae and ginko biloba L. A member of the botanical group Ginkgo is a substance that is capable of providing a similar physiological effect(s) as that provided by ginkgo in the compositions of the invention, and is preferably selected from a group comprising Ginkgo bilobae; Ginkgo bilobae L; Salisburia adianthifolia; Salisburia macrophylla; Salisburia adiantifolia; Salisburia adianthifolia.

“Chuanxiong,” as used herein, refers to extracts of chuanxiong. It is also known as radix ligustici chuanxiong and ligusticum chuanxiong hort. A member of the botanical group Ligusticum is a substance that is capable of providing a similar physiological effect(s) as that provided by chuanxiong in the compositions of the invention, and is preferably selected from a group comprising Radix ligustici chuanxiog; Ligusticum chuanxiong Hort; Ligusticum wallichii Franch; Cnidium officinale Makino;Ligusticum chuanxiong (S. H. Qiu. et al); Ligusticum sinense; Ligusticum chinensis; Ligustrum indicum; Ligustrum microcarpum; Ligustrum sinense; Ligustrum sinense var. stauntonii; Ligustrum stauntonii; Ligustrum villosum; Phillyrea indica.

“Shichangpu,” as used herein, refers to extracts of shichangpu. It is also known as rhizoma acori grami and acorus gramineus soland. A member of the botanical group Acorus gramineus is a substance that is capable of providing a similar physiological effect(s) as that provided by shichangpu in the compositions of the invention, and is preferably selected from a group comprising Rhizoma acori grami; Acorus gramineus soland; Acorus gramineus soland var.pusillus (sieb)Engl.; Acorus calamua L.; Acorus calamus Lour.

“Vitamin B-1,” as used herein, refers to the thiamine member of the Vitamin B series or complex.

“Vitamin B-2,” as used herein, refers to the riboflavin member of the Vitamin B series or complex.

“Vitamin B-6,” as used herein, refer to the pyridoxine member of the Vitamin B series or complex.

“Vitamin B-12,” as used herein, refers to the cobalamin member of the Vitamin B series or complex.

“Vitamin C,” as used herein, refers to ascorbic acid and salts thereof.

“Beta-carotene,” as used herein, refers to the form(s) of Vitamin A known to persons of skill in the art as such.

“Vitamin E,” as used herein, refers to D alpha-tocopherol, preferably in its succinate form.

“Magnesium,” as used herein, refers to the form(s) of the mineral known to persons of skill in the art to be therapeutically effective in the body of the individual. It is preferably provided as magnesium stearate NT.

“Inositol,” as used herein, refers to myoinositol or mesoinositol that is a component of the vitamin B complex.

“Quercetin,” as used herein, refers to the substance known in the art to result from hydrolysis of quercitron. Quercitron can be obtained from sources known in the art, such as quercitron bark.

“Manganese,” as used herein, refers to the form(s) of the mineral known to persons of skill in the art to be therapeutically effective in the body of the individual. It is preferably provided as manganese gluconate.

“Zinc,” as used herein, refers to the form(s) of the mineral known to persons of skill in the art to be therapeutically effective in the body of the individual. It is preferably provided as zinc gluconate.

“Copper,” as used herein, refers to the form(s) of the mineral known to persons of skill in the art to be therapeutically effective in the body of the individual. It is preferably provided as copper gluconate.

“Molybdenum,” as used herein, refers to the form(s) of the mineral known to persons of skill in the art to be therapeutically effective in the body of the individual. It is preferably provided as molybdenum aminoate.

“Selenium,” as used herein, refers to the form(s) of the mineral known to persons of skill in the art to be therapeutically effective in the body of the individual. It is preferably provided as selenium aminoate.

“Magnesium,” as used herein, refers to the form(s) of the mineral known to persons of skill in the art to be therapeutically effective in the body of the individual. It is preferably provided either as magnesium oxide or magnesium stearate NF, as specified in this specification.

“Extract,” as used herein, refers to the substances obtained from the specified source plant, or parts thereof (for e.g., root, bark, leaves). Any method of extraction that yields extracts that retain the biological activity of the substances contained in the extract source can be used to produce extracts used in this invention. Preferably, the ingredients of the compositions of the present invention are extracted as an aqueous solution. The extraction is preferably performed under conditions of high pressure, preferably from 0.5 to 12 bar, more preferably 1 to 10 bar, most preferably 3 to 7 bar, and preferably at elevated temperatures (preferably within a range of 15° C. to 120° C., more preferably 30° C. to 100° C., most preferably 45° C. to 75° C.). The extract is preferably treated to yield a form suitable for mixing of two or more substances. The form is preferably a dried powder. The powder form is yielded from preferably at least about a 1:10, more preferably at least about a 1:8, most preferably at least about a 1:5 concentrate of the starting solution. Concentration to powder form is preferably achieved by evaporation to yield a dried powder form. The extracts used in this invention can also be obtained from commercial sources such as Sun Ten Laboratories (Irvine, Calif.), Qualiherb (Cerritos, Calif.), Mayway (Oakland, Calif.), Ming Tong Herb (Oakland, Calif.) and Acta (Sunnyvale, Calif.). It is understood that any method or conditions known in the art to yield extracts comparable in therapeutic effectiveness or effectiveness in enhancing brain function to those produced by the preceding preferred extraction method can be used for the purposes of this invention.

Formulation of the compositions

Each substance contained in the compositions provided by this invention is provided in an amount that lies within specific quantitative ranges herein disclosed to be effective for treating sub-optimal brain function conditions or enhancing brain function.

According to the present invention, an effective amount of a composition comprises preferably from 12 mg to 200 mg, more preferably 25 mg to 150 mg, most preferably 50 mg to 100 mg of a member of the botanical group Angelica (such as dangqui); preferably 20 mg to 300 mg, more preferably 60 mg to 200 mg, most preferably 100 mg to 150 mg of a member of the botanical group Codonopsis (such as dangsen); preferably 12 mg to 200 mg, more preferably 25 mg to 150 mg, most preferably 50 mg to 100 mg of a member of the botanical group Jujuba (such as suanzaore); and/or preferably 20 mg to 300 mg, more preferably 60 mg to 200 mg, most preferably 100 mg to 150 mg of a member of the botanical group Schisandra (such as wuweizi).

In the various embodiments of the invention, the compositions further comprise preferably from 20 mg to 3000 mg, more preferably 200 mg to 2000 mg, most preferably 500 mg to 1000 mg of a member of the botanical group Panax (such as renshen); preferably 40 mg to 600 mg, more preferably 120 mg to 500 mg, most preferably 250 mg to 400 mg of a member of the botanical group Astragalus (such as huangqi); preferably 25 mg to 400 mg, more preferably 80 mg to 300 mg, most preferably 120 mg to 200 mg of a member of the botanical group Senticosus (such as ciwujija); preferably 25 mg to 400 mg, more preferably 80 mg to 300 mg, most preferably 120 mg to 200 mg of a member of the botanical group Gynostemma (such as jiaogulan); preferably 5 mg to 100 mg, more preferably 25 mg to 75 mg, most preferably 50 mg to 60 mg of a member of the botanical group Polygala (such as yuanzhi); preferably 12 mg to 200 mg, more preferably 25 mg to 150 mg, most preferably 50 mg to 100 mg of a member of the botanical group Citrus reticulata (such as chenpi); preferably 12 mg to 200 mg, more preferably 25 mg to 150 mg, most preferably 50 mg to 100 mg of a member of the botanical group Zingiberis (such as ginger); preferably 12 mg to 200 mg, more preferably 25 mg to 150 mg, most preferably 50 mg to 100 mg of a member of the botanical group Glycyrrhiza (such as gancao); 12 mg to 200 mg, more preferably 25 mg to 150 mg, most preferably 50 mg to 100 mg of a member of the botanical group ginkgo (such as ginkgo bilobae); preferably 80 mg to 1200 mg, more preferably 200 mg to 1000 mg, most preferably 350 mg to 750 mg of a member of the botanical group Ligusticum (such as chuanxiong); preferably 12 mg to 200 mg, more preferably 25 mg to 150 mg, most preferably 50 mg to 100 mg of a member of the botanical group Acorus gramineus (such as shichangpu); preferably 2.5 mg to 75 mg, more preferably 10 mg to 65 mg, most preferably 20 mg to 40 mg vitamin B-1; preferably 2.5 mg to 75 mg, more preferably 10 mg to 65 mg, most preferably 20 mg to 40 mg vitamin B-2; preferably 2.5 mg to 75 mg, more preferably 10 mg to 65 mg, most preferably 20 mg to 40 mg vitamin B-6; preferably 1.2 mg to 20 mg, more preferably 5 mg to 15 mg, most preferably 7.5 mg to 12.5 mg vitamin B-12; preferably 150 mg to 2000 mg, more preferably 750 mg to 1500 mg, most preferably 1000 mg to 1250 mg vitamin C; preferably 2.5 mg to 75 mg, more preferably 10 mg to 65 mg, most preferably 20 mg to 40 mg beta-carotene; preferably 10 mg to 175 mg, more preferably 50 mg to 125 mg, most preferably 75 mg to 100 mg vitamin E; preferably 22 mg to 300 mg, more preferably 55 mg to 200 mg, most preferably 100 mg to 150 mg magnesium oxide; preferably 2.5 mg to 75 mg, more preferably 10 mg to 65 mg, most preferably 20 mg to 40 mg inositol; preferably S mg to 10 mg, more preferably 6.5 mg to 9 mg, most preferably 7.5 mg to 8 mg quercetin; preferably 0.5 mg to 10 mg, more preferably 2 mg to 8 mg, most preferably 4 mg to 6 mg manganese; preferably 22 mg to 275 mg, more preferably 75 mg to 200 mg, most preferably 125 mg to 175 mg zinc; preferably 1.8 mg to 25 mg, more preferably 5 mg to 20 mg, most preferably 10 mg to 15 mg copper; preferably 2.5 mg to 75 mg, more preferably 10 mg to 65 mg, most preferably 20 mg to 40 mg molybdenum; preferably 2.5 mg to 75 mg, more preferably 10 mg to 65 mg, most preferably 20 mg to 40 mg selenium; and/or preferably 8 mg to 120 mg, more preferably 20 mg to 100 mg, most preferably 50 mg to 75 mg magnesium stearate NF.

Selection of suitable members of a particular botanical group to be included in a composition can be achieved using methods known in the art. For example, a suitable member of the botanical group Angelica would be expected to be capable of providing a similar physiological effect(s) as that provided by dangqui in a composition of the invention. Such a member can be selected based on, for example, whether it is known, shown and/or suspected to possess said similar physiological effect(s). Thus, for example, a determination of whether a candidate substance can be a member of the botanical group Angelica can be done based on, for example, a similar pharmacological or medicinal classification for both the candidate substance and dangqui. However, the activity and/or function provided by a particular substance, such as dangqui, need not be identified or specified. A determination of whether a candidate substance can be a member of a particular botanical group, for example the Angelica group, can also be empirical, for example, by substituting said candidate substance for dangqui in a composition, and assessing the relevant therapeutic effect(s) of the composition. Such a determination can be done using methods and techniques known in the art.

According to this invention, the compositions can be formulated in whatever form that retains the efficacy of the composition for treating sub-optimal brain function conditions or enhancing brain function. Preferably, the compositions are packaged in the form of capsules. The capsules are preferably of size “0”, “000”, “1”, “2”, “3” or “4.” A preferred method for packaging into capsules involves mixing substances (extracts, vitamin and minerals) that are preferably in powder form. The substances are preferably mixed to at least 30%, more preferably to at least 60%, even more preferably to at least 90% mixture consistency, and most preferably to homogeneity. The substances in powder form are provided in the initial mixture at ratios according to the effective quantities disclosed above. Methods for mixing the substances are known in the art, including, but not limited to, stirring, agitation or vibration achieved manually or through the aid of a machine. A preferred mixing machine is a V-mixer, preferably of 100 to 1400-liter size, more preferably of 150 to 1300-liter size, and most preferably of 200 to 1200-liter size. Preferably, the resulting powder mixture is filtered to screen out particulates (i.e., anything that a person of skill in the art would recognize to be larger than powder size). A preferred filter is a 1/20-inch particle size filter. Preferably, the filtered mixture is packaged into capsules according to the weight desired for each capsule. Preferably, the capsule is of size “00”. The weight of mixture per capsule is preferably from 5 mg to 1000 mg, more preferably 100 mg to 800 mg, even more preferably 400 mg to 700 mg. It is understood that other physical forms of the compositions of this invention suitable for administration to an individual can also be used, including, for example, tablets, salves or liquids, as long as the compositions can be delivered to the target tissues in the body where the compositions in the preferred form described above exert their effects.

The ingredients of the compositions can be mixed with pharmaceutically acceptable solvents, excipients and/or filler substances. These materials are known in the art, and are described in sources such as Remington's Pharmaceutical Sciences, 18th Edition, Mack Publishing (1990).

Administration of compositions

Compositions in any of the physical forms described above can be administered by any method known to one of skill in the art, but oral administration is preferred. The compositions are preferably administered in capsule form.

An effective amount of a composition is provided preferably in from 1 to 8 administrations, more preferably in from 2 to 6 administrations, and most preferably in from 3 to 5 administrations. Administration of an effective amount is preferably completed within 24 hours. A composition can be ingested alone, or with any other substance, such as a liquid, that aids ingestion of the compositions. Ingestion of the compositions can be before or after food consumption.

### EXAMPLES

### Example 1

An Illustrative Example of the Formulation of A Single “00” Capsule. and the Production Thereof

A composition shown to be effective for treating sub-optimal brain function conditions or enhancing brain functions contained substances in the indicated quantities as listed in Table 1.

TABLE 1

| SUBSTANCE [commercial source] | AMOUNT (mg) |
|---|---|
| Dangqui [Mayway, Oakland, CA] | 16.7 |
| Dangsen [Qualiherb, Cerritos, CA] | 25 |
| Suanzaore [Qualiherb, Cerritos, CA] | 16.7 |
| Wuweizi [Mayway, Oakland, CA] | 25 |
| Renshen [Qualiherb, Cerritos, CA] | 25 |
| Huangqi [Mayway, Oakland, CA] | 50 |
| Ciwujia [Mayway, Oakland, CA] | 33.3 |
| Jiaogulan [Mayway, Oakland, CA] | 33.3 |
| Yuanzhi [Qualiherb, Cerritos, CA] | 8.3 |
| Chenpi [Qualiherb, Cerritos, CA] | 16.7 |
| Ginger [Qualiherb, Cerritos, CA] | 16.7 |
| Gancao [Qualiherb, Cerritos, CA] | 16.7 |
| Ginkgo [Mayway, Oakland, CA] | 16.7 |
| Chuanxiong [Qualiherb, Cerritos, CA] | 100 |
| Shichangpu [Qualiherb, Cerritos, CA] | 16.7 |
| Vitamin C [Acta, Sunnyvale, CA] | 183.3 |
| Beta-carotene [Acta, Sunnyvale, CA] | 5.0 |
| Vitamin E (D Alpha-Tocopherol | 14.1 |
| succinate @ 1.185 iu/mg) [Acta, | |
| Sunnyvale, CA] | |
| Vitamin B-1 (Thiaminee) [Acta, | 5.0 |
| Sunnyvale, CA] | |
| Vitamin B-2 (Riboflavin) [Acta, | 5.0 |
| Sunnyvale, CA] | |
| Vitamin B-6 (Pyridoxine) [Acta, | 5.0 |
| Sunnyvale, CA] | |
| Vitamin B-12 (Cyanocobalamin) [Acta, | 1.7 |
| Sunnyvale, CA] | |
| Magnesium oxide [Acta, Sunnyvale, CA] | 27.8 |
| Inositol [Acta, Sunnyvale, CA] | 5.0 |
| Manganese gluconate [Acta, Sunnyvale, | 0.76 |
| CA] | |
| Zinc gluconate [Acta, Sunnyvale, CA] | 25.64 |
| Copper gluconate [Acta, Sunnyvale, CA] | 2.38 |
| Molybdenum aminoate [Acta, Sunnyvale, | 5.0 |
| CA] | |
| Selenium aminoate [Acta, Sunnyvale, | 5.0 |
| CA] | |
| Magnesium stearate NF [Acta, 10.0 | |
| Sunnyvale, CA] | |

Capsules containing the composition above were manufactured according to the method used by the commercial manufacturer, Acta (Sunnyvale, Calif.). Briefly, the substances listed above, in powder form and obtained from the commercial sources indicated, were mixed in input amounts in accordance to the ratio of the substances in the composition as a whole. Mixing was accomplished with a V-mixer, grinding for 15 to 30 minutes, at a speed of 15 to 30 rpm (rounds per minute), to produce a homogenous mixture of the input substances. Particulates (non-powder forms) were then filtered out with a 1/20-inch particle size filter that separated particulates from the powder. 725.6 mg of the filtered mixture was then packaged into each size “00” capsule.

### Example 2

A First Illustrative Example of Treatment of Sub-optimal Brain Function Conditions or Enhancement of Brain Functions

Individuals complaining of sub-optimal brain function conditions or a need to enhance their brain function level are asked to fill out a questionnaire. The questionnaire requires the individuals to rate their brain function on a scale of, for example, 1 to 6, 1 being low level of brain function and 6 being excellent level of brain function. Individuals are instructed to assess mental or brain-related conditions such as memory, ability to concentrate, mental fatigue, irritability, ill-temper, quality of sleep (e.g., insomnia), existence or degree of depression, and/or absentmindedness as indicative of their level of brain function.

The individuals are daily administered at least 2 of the capsules exemplified in Example 1. Administration can be in one ingestion or spread over a 24-hour period, before or after a meal, with water or any liquid that helps swallowing of the capsules.

The individuals are asked to rate their level of brain function on the 1 to 6 scale at least once daily for at least 3 days.

Improvement or enhancement of brain function is indicated by an increase in the brain function ratings from the rating prior to administration of the capsules of Example 1.

### Example 3

A Second Illustrative Example of Treatment of Sub-optimal Brain Function Condition or Enhancement of Brain Functions

A randomized, double-blinded and controlled study is performed. The control group uses a sugar pill placebo. Dosage amount and schedule are 1 to 6 capsules (of the composition of Example 1) daily. At least 20 individuals are tested, randomly assigned in approximately equal number to the treatment (administered the composition of Example 1) and control groups. Individuals are evaluated on their mental or brain-related conditions such as memory, ability to concentrate, mental fatigue, irritability, ill-temper, quality of sleep (e.g., insomnia), existence or degree of depression, and/or absentmindedness as a measure of their brain function level.

The study is carried out for at least 1 week. During the treatment course, a dosage amount selected from the range of 1 to 6 capsules is administered to each individual once or multiple times daily, not exceeding 6 capsules per day. Capsules are administered in between or after meals.

A clinical questionnaire is used to evaluate the individual's brain function level. A clinical coordinator and/or physician evaluates the individual's brain function, and fills out the questionnaire. Evaluations can be performed daily, or more or less frequently depending on statistical or clinical (ability to detect or track symptomatic improvements) need.

Assessment of treatment or enhancement of brain function is divided into 4 grades: (1) clinical efficacy, as defined as achievement of normal brain function in individuals suffering from sub-optimal brain function prior to initiation of the study, or enhancement to the highest rating of brain function; (2) significant efficacy, as defined as significant improvement in brain function (e.g., improvement of more than 3 points on any one of brain function scoring scales); (3) efficacy, as defined as partial improvement in brain function (e.g., improvement of more than 1 point on any one of brain function scoring scales); and (4) non-efficacy, as defined as no improvement in brain function.

Although the foregoing invention has been described in some detail by way of illustration and example for purposes of clarity of understanding, it will be apparent to those skilled in the art that certain changes and modifications can be practiced. Therefore, the description and examples should not be construed as limiting the scope of the invention, which is delineated by the appended claims.

1. A composition for treating sub-optimal brain function or enhancing brain function comprising 2 substances selected from the group consisting of a member of the botanical group Angelica, a member of the botanical group Codonopsis, a member of the botanical group Jujuba and a member of the botanical group Schisandra.

2. A composition for treating sub-optimal brain function or enhancing brain function comprising 3 substances selected from the group consisting of a member of the botanical group Angelica, a member of the botanical group Codonopsis, a member of the botanical group Jujuba and a member of the botanical group Schisandra.

3. A composition for treating sub-optimal brain function or enhancing brain function comprising a member of the botanical group Angelica, a member of the botanical group Codonopsis, a member of the botanical group Jujuba and a member of the botanical group Schisandra.

4. A composition selected from the group consisting of the compositions of claims 1 to 3, further comprising a substance selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

5. A composition selected from the group consisting of the compositions of claims 1 to 3, further comprising 2 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

6. A composition selected from the group consisting of the compositions of claims 1 to 3, further comprising 3 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

7. A composition selected from the group consisting of the compositions of claims 1 to 3, further comprising 4 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

8. A composition selected from the group consisting of the compositions of claims 1 to 3, further comprising 5 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

9. A composition selected from the group consisting of the compositions of claims 1 to 3, further comprising 6 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramnineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

10. A composition selected from the group consisting of the compositions of claims 1 to 3, further comprising 7 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6. vitamin B-12, magnesium oxide, quercetin, inositol, manganese, zinc, copper, molybdenum, selenium and magnesium stearate NF.

11. A composition selected from the group consisting of the compositions of claims 1 to 3, further comprising 8 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

12. A composition selected from the group consisting of the compositions of claims 1 to 3, further comprising 9 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

13. A composition selected from the group consisting of the compositions of claims 1 to 3, further comprising 10 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

14. A composition selected from the group consisting of the compositions of claims 1 to 3, further comprising 11 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

15. A composition selected from the group consisting of the compositions of claims 1 to 3, further comprising 12 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

16. A composition selected from the group consisting of the compositions of claims 1 to 3, further comprising 13 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

17. A composition selected from the group consisting of the compositions of claims 1 to 3, further comprising 14 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF..

18. A composition selected from the group consisting of the compositions of claims 1 to 3, further comprising 15 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

19. A composition selected from the group consisting of the compositions of claims 1 to 3, further comprising 16 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

20. A composition selected from the group consisting of the compositions of claims 1 to 3, further comprising 17 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

21. A composition selected from the group consisting of the compositions of claims 1 to 3, further comprising 18 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

22. A composition selected from the group consisting of the compositions of claims 1 to 3, further comprising 19 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

23. A composition selected from the group consisting of the compositions of claims 1 to 3, further comprising 20 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

24. A composition selected from the group consisting of the compositions of claims 1 to 3, further comprising 21 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

25. A composition selected from the group consisting of the compositions of claims 1 to 3, further comprising 22 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

26. A composition selected from the group consisting of the compositions of claims 1 to 3, further comprising 23 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

27. A composition selected from the group consisting of the compositions of claims 1 to 3, further comprising 24 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

28. A composition selected from the group consisting of the compositions of claims 1 to 3, further comprising 25 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

29. A composition selected from the group consisting of the compositions of claims 1 to 3, further comprising 26 substances selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulate, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

30. A composition selected from the group consisting of the compositions of claims 1 to 3, further comprising a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.

31. The composition of any of claims 1-3, wherein the member of the botanical group Angelica is dangqui, the member of the botanical group Codonopsis is dangsen, the member of the botanical group Jujuba is suanzaore and the member of the botanical group Schisandra is wuweizi.

32. The composition of claim 4, wherein the member of the botanical group Angelica is dangqui, the member of the botanical group Codonopsis is dangsen, the member of the botanical group Jujuba is suanzaore, the member of the botanical group Schisandra is wuweizi, the member of the botanical group Panax is renshen, the member of the botanical group Astragalus is huangqi, the member of the botanical group Senticosus is ciwujia, the member of the botanical group Gynostemma is jiaogulan, the member of the botanical group Polygala is yuanzhi, the member of the botanical group Citrus reticulata is chenpi, the member of the botanical group Zingiberis is ginger, the member of the botanical group Glycyrrhiza is gancao, the member of the botanical group ginkgo is ginkgo, the member of the botanical group Ligusticum is chuanxiong, and the member of the botanical group Acorus gramineus is shichangpu.

33. A method for treating sub-optimal brain function or enhancing brain function comprising administering to the individual an effective amount of a composition selected from the group consisting of the compositions of claims 1 to 3.

34. A method of making a composition for treating sub-optimal brain function or enhancing brain function, said method comprising combining at least two substances selected from the group consisting of a member of the botanical group Angelica, a member of the botanical group Codonopsis, a member of the botanical group Jujuba and a member of the botanical group Schisandra.

35. The method of claim 34, wherein said at least two substances are -combined with at least one substance selected from the group consisting of a member of the botanical group Panax, a member of the botanical group Astragalus, a member of the botanical group Senticosus, a member of the botanical group Gynostemma, a member of the botanical group Polygala, a member of the botanical group Citrus reticulata, a member of the botanical group Zingiberis, a member of the botanical group Glycyrrhiza, a member of the botanical group ginkgo, a member of the botanical group Ligusticum, a member of the botanical group Acorus gramineus, vitamin C, beta-carotene, vitamin E, vitamin B-1, vitamin B-2, vitamin B-6, vitamin B-12, magnesium oxide, quercetin, inositol, manganese, zinc, copper, molybdenum, selenium and magnesium stearate NF.
